(19) United States
(12) Patent Application Publication
(10) Pub. No.: US 2009/0292015 A1
(43) Pub. Date: Nov. 26, 2009

(54) STABLE HIGH VITAMIN C CONTENT POLYOL-IN-OIL EMULSIFIED SYSTEM AND ITS PREPARATION

(75) Inventors: Haizhou Zhang, Shanghai (CN); Jingya Dai, Shanghai (CN); Jiali Zou, Shanghai (CN); Zhiheng Gao, Shanghai (CN)

Correspondence Address: SCULLY SCOTT MURPHY & PRESSER, PC, 400 GARDEN CITY PLAZA, SUITE 300, GARDEN CITY, NY, 11530, US

(73) Assignee: EVONIK GOLDSCHMIDT GMBH, Essen (DE)

(21) Appl. No.: 12/432,181

(22) Filed: Apr. 29, 2009

(30) Foreign Application Priority Data
May 20, 2008 (CN) 200810100726.7

(51) Int. Cl. A61K 31/375

(52) U.S. Cl. 514/474

(57) ABSTRACT

The present invention is directed to a polyol-in-oil composition used to transport and stabilize Vitamin C. The composition includes 0.5 wt % to 5 wt % of at least one emulsifier, 0.1 wt % to 5 wt % of at least one stabilizer, 0.01 wt % to 20 wt % of Vitamin C, 10 wt % to 40 wt % of at least one oil component and 40 wt % to 80 wt % of at least one polyol, each based on the mass of the whole composition, wherein the proportion of water in the whole composition is less than 1 wt % and wherein Vitamin C is dissolved in the polyols and the use of this composition.

### FIELD OF THE INVENTION

The present invention relates to a stable high Vitamin C content polyol-in-oil emulsified system and its preparation method. The composition of the present invention can be widely applied in the area of personal care products.

### BACKGROUND

Vitamin C is well known for its various biological functions, such as the stimulation of collagen synthesis, the strengthening of skin tissue against external attack (UV radiation, pollution), depigmentation, and anti free radicals. However, due to its alpha-keto lactone structure, Vitamin C is very sensitive to the influence of environmental parameters such as, for example, water, light, oxygen, and heavy metals. Degradation of vitamin C in a water solution occurs over time, which limits the application of vitamin C in cosmetic products.

CN1466939A teaches that Vitamin C stability could be improved by the combination of propylene glycol or glycerin with water. As a matter of fact, U.S. Pat. No. 4,983,382, U.S. Pat. No. 5,981,578, U.S. Pat. No. 5,736,567 and U.S. Pat. No. 6,020,367 also disclose this information. Although a certain effect is detectable, the product color still deteriorates to yellow.

CN1223567A records that the solubility of Vitamin C in non-water media is relatively low and that the active concentration of Vitamin C in such a system is also low. The Chinese document further mentions that the combination of dimethyl isosorbide, dimetihicone and alcohols can improve the stability of Vitamin C, but this system is not an emulsion, all these components are just mixed, thus it may not be stable with time, if used as a cosmetic product. Also, the Vitamin C in this system is dispersed as crystals, but not dissolved. Therefore this system can not be a good Vitamin C transportation system for skin application.

CN1251773A relates to the use of a silicone gum to stabilize ascorbic acid or one of its esters or salts in a topical composition in which dimethicone copolyol, ABIL® EM 90, Cyclopentasiloxane and glycerin are used. Such a composition however also contains some water.

A number of references mention that ABIL® EM 90 could be used as the emulsifier of a W/O system, in which the water could include ethanol or other polyols, e.g., CN1520282A, CN1260166A, CN1199608A, CN1251773A, CN1466939A, CN1592603A, CN1223567A, CN1370062A, CN1223569A, CN1328817A and CN1471903A. CN1520282A clearly teaches ABIL® EM 90 could be used with polyols in a silicone system.

Hydrophobic silica is always used as a stabilizer in a W/O system, e.g., CN1471903A and CN1173125A. CN1471903A relates to a foundation in the form of a W/O emulsion comprising a fatty phase, an aqueous phase, at least one surfactant chosen from C8-C22 alkyl dimethicone copolyols, at least one other surfactant chosen from dimethicone copolyols, and hydrophobic coated pigments. In its description, one of the C8-C22 alkyl dimethicone copolyols could be ABIL® EM 90. The system could contain a stabilizer such as AEROSIL® R 812, other hydrophilic components such as propylene glycol and glycerin, and active components such as Vitamin C.

In WO 2006/012723, a polyol/oil-phase composition is described, where the Vitamin C is dissolved in the polyol phase at elevated temperatures and after cooling down to room temperature, the oil phase is added to the polyol phase. One drawback of this prior art composition is the need of a relatively large amount of emulsifier. Therefore, an optimized system is still required, which can sufficiently dissolve and stabilize Vitamin C, have a comfortable skin feeling when applied, and be stable with shelf time. Also, a friendly industrial process is needed.

### SUMMARY OF THE INVENTION

The present invention provides a composition which is capable of dissolving a high amount of Vitamin C without exhibiting one or more of the disadvantages of the prior art.

Surprisingly the applicants of the present invention have found that an emulsion system comprising a polyol-in-oil composition can be obtained by choosing an optimized emulsifier.

Specifically, the present invention provides an emulsion system comprising a polyol-in-oil composition that includes 0.5 wt % to 5 wt % of at least one emulsifier, 0.1 wt % to 5 wt/o of at least one stabilizer, 0.01 wt % to 20 wt % of Vitamin C, 10 wt % to 40 wt % of at least one oil component and 40 wt % to 80 wt % of at least one polyol, each based on the mass of the whole composition, wherein the amount of water in the whole composition is less than 1 wt % and wherein the Vitamin C is dissolved in the polyols.

The inventive emulsion has a traditional white and shiny appearance, and the Vitamin C inside is dissolved in polyols, not in water, which lowers the contact of oxygen and metal ions with Vitamin C.

Because the content of oxygen and metal ions in polyols is much lower than that in water, the stability of dissolved Vitamin C can be dramatically increased in this system due to less oxidation. As the system of the invention is, or can be essentially free of water, it is not necessary to use preservatives, and therefore it is less harmful to the skin. The optimized emulsifier Cetyl PEG/PPG-10/1 dimethicone (ABIL® EM 90) together with optimized hydrophobic aerosil makes any cream made by this system have a shinny and white appearance. This cream is very stable with time and can easily be used in personal care field.

Furthermore, the polyols particles (droplets) are encapsulated in the emulsion system by oil components, thus the risk of oxidation is decreased further.

The present invention solves the problem that Vitamin C is quickly degraded in a normal cosmetics product. This invention also provides an innovative emulsified system which is a waterless polyol-in-oil emulsion and can be a stable cosmetic formula.

Although the concept of polyols in oil is proposed in CN1520282A, it is only used for a pre-composition which is part of the final formula. Also, in CN1520282A, the weight ratio of emulsifiers to polyols is from 100:1 to 2:8. If the content of polyols is close to 50 wt %, the emulsifier quantity would be around 13 wt %, which is obviously too high for a cosmetics formula.

### BRIEF DESCRIPTION OF THE DRAWINGS

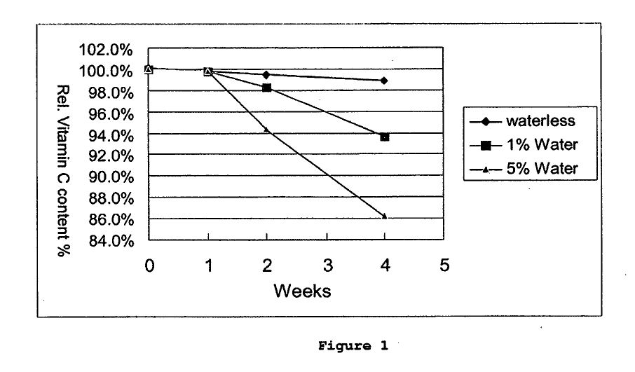

The sole figure, i.e., FIG. 1, is a graph of relative Vitamin C content in % vs. weeks for a waterless system, a system including 1% water and a system including 5% water as described in example 1 and comparative examples 1 and 2.

### DETAILED DESCRIPTION OF THE INVENTION

The polyol-in-oil composition (e.g., polyol-in-oil emulsion system) according to the present invention, which is useful to transport and stabilize Vitamin C, includes from 0.5 wt % to 5 wt % (% by weight), preferably from 1 wt % to 4.5 wt % and more preferably from 2.5 wt % to 3.5 wt % of at least one emulsifier, from 0.1 wt % to 5 wt %, preferably from I wt % to 4 wt % of at least one stabilizer, from 0.01 wt % to 20 wt %, preferably from 5 wt % to 15 wt % ascorbic acid (Vitamin C), from 10 wt % to 40 wt % of at least one oil component and from 40 wt % to 80 wt %, preferably from 45 wt % to 70 wt % and more preferably from 50 wt % to 65 wt % of at least one polyol, each based on the mass of the whole composition, wherein the proportion of water in the whole composition is less than 1 wt %, preferably less than 0.1 wt %, and wherein Vitamin C is dissolved in the polyols.

Preferably the amount of water in the composition is essentially 0 wt %. More preferably no measurable amount of water is contained in the composition of the invention. The optimized content of Vitamin C in the inventive composition is from 10 wt % to 15 wt %.

The emulsifier(s) used in the polyol-in-oil composition of the present invention are preferably selected from cetyl PEG/PPG-10/1 dimethicone (for example ABIL® EM 90 of Evonik Goldschmidt GmbH) and a combination of poly glyceryl alkyl esters. A preferred combination of poly glyceryl alkyl esters is selected from the combination of poly glyceryl alkyl esters containing 1 portion by weight of polyglyceryl-4 diisostearate/polyhydroxystearate/sebacate and 0.25 to 4 portion by weight of polyglyceryl-4 stearate, and from the combination containing 1 portion by weight of diisostearoyl polyglyceryl-3 dimer dilinoleate and 0.25 to 4 portion by weight of polyglyceryl-4 stearate. The molecular weight of the preferred cetyl PEG/PPG-10/1 dimethicone (Evonik Goldschmidt GmbH, ABIL® EM 90) is about 14000 g/mol.

The structure of the preferred cetyl PEG/PPG-10/1 dimethicone is given in formula (I),

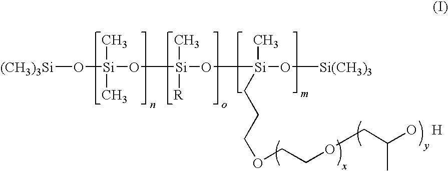

wherein R is a cetyl group, x=10, y=1, n=1 to 200, o=1 to 100, and m=1 to 40.

The stabilizers contained in the composition of the present invention are preferably selected from hydrophobic bentonite or hydrophobic silica, especially hydrophobic pyrogenic silica (available under the trade name AEROSIL® from Evonik Degussa GmbH). Preferably the stabilizers employed in the present invention are selected from nanometer sized or micrometer sized particles of hydrophobic bentonite or hydrophobic silica. The BET value of a preferred pyrogenic silica is from 200 to 300m²/g. The methanol wettability of a preferred pyrogenic silica is from 50 to 70. Preferred stabilizers are AEROSIL® R 812 S from Evonik Degussa GmbH (hydrophobic pyrogenic silica) or bentonite stabilizer a hydrophobic bentonite from Elementis Specialties available under the trade name Bentone® 38V. The above mentioned pyrogenic silica that can be employed in this invention is preferably made by a pyrolysis method.

A preferable oil component of the composition of the present comprises silicones, organic modified silicones, fatty hydrocarbons, fatty alcohols and/or fatty esters. Preferred silicones and organic modified silicones are chosen from cyclomethicone (Dow Corning® 345), dimethicone (Goldschmidt, ABIL® 350) or silicone copolymer (Dow Corning 9040) or a combination thereof The fatty hydrocarbons, fatty alcohols, fatty esters are preferably selected from paraffin oil, polyisobutene, cetyl alcohol, stearyl alcohol or their mixture, octyldodecanol, cetearyl isononanoate, cetyl ethylhexanoate, cetyl ricinoleate, caprylic/capric triglyceride, decyl oleate, diethyihexyl carbonate, decyl cocoate, isocetyl palmitate, cetearyl ethylhexanoate, isopropyl myristate, myristyl myritate, octyl palmitate, octyl stearate, isopropyl palmitate, triisostearin, steryl heptanoate, 12-15 alkyl benzoate, PPG-15 stearyl ether, PPG-14 butyl ether, PPG-3 myristyl ether or PPG-11 stearyl ether or mixtures thereof.

The polyols used in the present invention preferably are monohydric, dihydric, or trihydric alcohols, preferably comprising less than 5 carbons. More preferably the polyol is selected from glycerin, propylene glycol, 1,3-butylene glycol and ethanol or a mixture thereof. Conventional, monohydric alcohols and polyols are totally different materials. But in the present invention, for convenience, monohydric alcohols, especially ethanol, are taken as part of the polyols.

In some embodiments of the invention it is an advantage to add ethanol as a monohydric alcohol to the polyol-in-oil composition. The amount of mono alcohol, especially of ethanol in the composition of the invention is preferably from 0 wt % or 1 wt % to 10 wt %, more preferably from 2.5 wt % to 5 wt %.

The polyol-in-oil composition (emulsion system) of the present invention can be produced by any method known in the art Preferably the polyol-in-oil composition (emulsion system) of the present invention is prepared using the following processing steps:
a.) mixing all oil components, emulsifiers and stabilizers, preferably at room temperature (20° C.-30° C.), preferably under agitation to obtain a homogeneous phase,
b.) adding Vitamin C to one or more polyols and heating up this mixture up to a temperature above room temperature, preferably to a temperature of 90° C. or more, and more preferably to a temperature of from 90° C. to 100° C. until Vitamin C dissolves completely and a homogeneous mixture is obtained which is then brought down to room temperature, and
c.) adding the polyols phase obtained in step b.) into the oil phase obtained in step a.) preferably slowly and under stirring, more preferably at a stirring speed of from 400 to 600 rpm, and then homogenizing the mixture at higher stirring speed, preferably from 1000 to 16000 rpm, preferably for several minutes, more preferably from 15 to 30 minutes, to obtain the polyol-in-oil emulsion with a content of Vitamin C. In some embodiments of the invention, it is advantageous when one or more alcohols having a boiling point below the maximum process temperature used in step b.), preferably below 90° C. are added to the polyols mixture after the dissolution of the Vitamin C in the polyol phase in step b.) and before step c.). Preferably the polyol phase is cooled down to a temperature of 45° C. or less before the low boiling alcohol is added. The alcohol added is preferably ethanol. The amounts of ingredients preferably used in the process of the invention can directly be taken from the content of the ingredients of the composition of the invention, especially the preferred embodiments of the composition.

The polyols used at the beginning of step b.) are preferably selected from diols or polyols, more preferably selected from glycerin, propylene glycol and 1,3-butylene glycol or mixtures thereof.

Due to the above mentioned technical method, Vitamin C is successfully dissolved in the polyols phase which is also well encapsulated by oil components in the composition of the present invention. Because the solubility of oxygen in polyols or oil components is much lower than in water, the emulsifying system of the present invention extremely protects Vitamin C from oxygen, and therefore makes it more stable. Furthermore, the composition of the invention is essentially free of water to avoid the catalyzed oxidation by trace amounts of metal ions (i.e., iron ion, cupric ion) often present in water in traces.

Because the composition of the invention is (essentially) free of water it is not necessary to add any preservatives to the composition. It is therefore less harmful to the skin then those products comprising water and preservatives.

With the use of the preferred emulsifier Cetyl PEG/PPG-10/1 dimethicone (ABIL® EM 90) together with the preferred stabilizer hydrophobic pyrogenic silica a cream can be obtained having a shinny and white appearance. This cream is very stable over time and can easily be used in personal care field. Therefore one more aspect of the invention is a personal care product and especially a cosmetic product comprising a polyol-in-oil composition according to the invention. The personal care and cosmetic product can comprise one or more of the compounds known to be used in personal care and cosmetic products.

The personal care and cosmetic products according to the invention can, e.g., comprise at least one additional component chosen from the group consisting of
emollients
emulsifiers and surfactants
thickeners/viscosity regulators/stabilizers
UV/light screening agents
antioxidants
solids
pearlescent additives
insect repellents
preservatives
fragrances
colorants
biogenic active substances
care additives.

Preferred examples of these components are listed in DE 102005011785 and EP 2000124 which are incorporated by reference.

The invention is now described in detail by the following examples. The scope of the invention should not be bound to the embodiments of the examples but is given by the scope of the claims.

### EXAMPLE 1 AND COMPARATIVE EXAMPLES 1 AND 2

The composition of example 1 was prepared in accordance with the process of the invention described above:
a.) 25.4 wt % cyclomethicone (Dow Corning® 345), 5.5 wt % dimethicone (ABIL® 350), 3 wt % emulsifier (ABIL® EM 90), and 3 wt % stabilizer (AEROSIL R 812 S) were mixed at room temperature by stirring to obtain a homogeneous phase.
b.) 10.5 wt % Vitamin C were added into a mixture of polyols of 45 wt % Glycerin and 7.6 wt % propylene glycol. The polyol phase was heated up to about 90° C. and kept there for 30 to 45 minutes until the whole phase was clear and transparent.
c.) The polyols phase was added slowly the into the oil components phase obtained in step a.) at a stirring speed of about 5000 rpm. Then the whole system was mixed at a speed of from 1000 to 16000 rpm until a homogeneous cream was achieved.

The preparation processes for the comparative examples I and 2 were performed similar to that of the example 1. The difference was that different amount of water was added after step b.).

TABLE 1
Ingredients used to obtain the compositions of example 1 and
comparative examples 1 and 2

| | | | Comparative | Comparative |
|---|---|---|---|---|
| | | Example 1 | example 1 | example 2 |
| | Ingredients | (wt %) | (wt %) | (wt %) |
| A | Cyclomethicone (Dow | 25.4 | 25.4 | 25.4 |
| | Corning ® 345) | | | |
| | Hydrophobic silica | 3 | 3 | 3 |
| | (AEROSIL R 812 S) | | | |
| | Cetyl PEG/PPG-10/1 | 3 | 3 | 3 |
| | dimethicone (ABIL ® EM | | | |
| | 90) | | | |
| | Dimethicone | | | |
| | (ABIL ® 350) | 5.5 | 5.5 | 5.5 |
| B | Propylene glycol | 7.6 | 7.6 | 7.6 |
| | Glycerin | 45 | 44 | 40 |
| | Vitamin C | 10.5 | 10.5 | 10.5 |
| | Water | 0 | 1 | 5 |

The content of Vitamin C was analyzed according to the method published in Chinese pharmacopoeias year 2000 version, second section, page 791 (ISDN: 7502527362, Chemical Industry Press): After step 3, the content of Vitamin C was analyzed to be 10.3 wt % in example 1. A part of the composition of example 1 was held at room temperature (20° C.-25° C.) for three months, and then the Vitamin C content was analyzed again. The result showed no Vitamin C degradation. Another part of the composition of example 1 was kept in a completely filled and closed bottle. This bottle was stored in an oven at a temperature of 45° C. for 4 months. The composition was analyzed after the end of this treatment. The Vitamin C content was still 97.1% of the original content, which means its degradation is less than 3%. The above mentioned samples were still stable and no separation, roughness and obvious color changing had been observed.

The contents of Vitamin C in comparative examples 1 and 2 were treated and analyzed under the same conditions as example 1. In FIG. 1, a graph shows the results of the stability measurement. It can easily be seen that the stability of Vitamin C is very sensitive to the water content: a content of 1 wt % of water in the composition of comparative example 1 at 50° C. gives a degradation of 6% after one month and this degradation has already caused an obvious color changing. A content of 5 wt % of water in the composition (comparative example 2) gives a degradation of 14% after one month.

The above test data demonstrates the perfect stability of Vitamin C in the composition of example 1. Furthermore, this composition also passed heat stability test (4 months in 45° C.), cold stability test (one month in −25° C.) and centrifugation test at 3000 rpm, 30 minutes, which shows its very good stability.

### EXAMPLE 2

The preparation process for preparing the composition of example 2 was performed similar to that of the example 1. The difference was that different ingredients (according to table 2) were used to obtain the oil components phase in step a.).

TABLE 2
Ingredients used to obtain the compositions of example 2

| | Ingredients | wt % |
|---|---|---|
| A | Cyclomethicone (Dow Corning ® 345) | 16.4 |
| | Silicone copolymer (Dow Corning ® 9040) | 10 |
| | Hydrophobic aerosil (AEROSIL R 812 S) | 2 |
| | Cetyl PEG/PPG-10/1 dimethicone (ABIL ® EM 90) | 3 |
| | Dimethicone (ABIL ® 350) | 5.5 |
| B | Propylene glycol | 7.6 |
| | Glycerin | 45 |
| | Vitamin C | 10.5 |

This composition also passes heat stability test (4 months in 45° C.), cold stability test (one month in −25° C.) and centrifugation test at 3000 rpm, 30 minutes, which shows its very good stability.

### EXAMPLES 3 AND 4

The preparation processes for preparing the compositions of examples 3 and 4 were performed similar to that of the example 1. The difference was that different ingredients (according to table 3) were used to obtain the oil components phase in step a.).

TABLE 3
Ingredients used to obtain the compositions of example 3 and 4

| | | Example 3 | Example 4 |
|---|---|---|---|
| | Ingredients | (wt %) | (wt %) |
| A | Cyclomethicone (Dow Corning ® 345) | 3 | 15 |
| | paraffin oil | 15.5 | 15.5 |
| | Hydrophobic aerosil (AEROSIL R 812 S) | 1.5 | 1.5 |
| | Polyglyceryl-4 diisostearate/ | 3 | |
| | spolyhydroxystearate/ebacate | | |
| | (Isolan ® GPS) | | |
| | Diisostearoyl polyglyceryl-3 dimer | | 3 |
| | dilinoleate (Isolan ® PDI) | | |
| | Polyglyceryl-4 isostearate (Isolan ® GI 34) | 2 | 2 |
| B | Propylene glycol | | 8 |
| | Glycerin | 60 | 45 |
| | Vitamin C | 15 | 10 |

The emulsifier mixture of Polyglyceryl-4 diisostearate/polyhydroxystearate/sebacate (Isolan® GPS) and Polyglyceryl-4 isostearate (Isolan® GI 34) obtained in example 3 showed a good stability. The emulsifier mixture of Diisostearoyl polyglyceryl-3 dimer dilinoleate (Isolan® PDI) and Polyglyceryl-4 isostearate (Isolan® GI 34) obtained in example 4 showed a shinny and white appearance and was also a stable system.

### EXAMPLE 5

The composition of example 5 was prepared in accordance with the process of the invention described above:
a.) 25.4 wt % cyclomethicone (Dow Corning® 345), 5.5 wt % dimethicone (ABIL® 350), 3 wt % emulsifier (ABIL® EM 90), and 3 wt % stabilizer (AEROSIL R 812 S) were mixed at room temperature (20-30° C.) by stirring to obtain a homogeneous phase.
b.) 10.5 wt % Vitamin C were added into a mixture of polyols of 21 wt % glycerin, 7.6 wt % propylene glycol and 20 wt % 1,3-butylene glycol. This polyol phase was heated up to about 95° C. and kept there for 30 to 45 minutes until the whole phase was clear and transparent. The phase was then cooled down to below 45° C. and then 4 wt % of ethanol was added with agitation.
c.) The polyols phase was added slowly to the oil components phase of step a.) at a speed of about 500 rpm and the whole system was then mixed at a speed of 1000-16000 rpm until a homogeneous cream was achieved.

The ingredients used in example 5 are given in table 4.

TABLE 4
Ingredients used to obtain the compositions of example 5

| | Ingredients | wt % |
|---|---|---|
| A | Cyclomethicone (Dow Corning ® 345) | 25.4 |
| | Hydrophobic aerosil (AEROSIL R 812 S) | 3 |
| | Cetyl PEG/PPG-10/1 dimethicone (ABIL ® EM 90) | 3 |
| | Dimethicone (ABIL ® 350) | 5.5 |
| B | Propylene glycol | 7.6 |
| | Glycerin | 21 |
| | 1,3-butylene glycol | 20 |
| | Ethanol | 4 |
| | Vitamin C | 10.5 |

While the present invention has been particularly shown and described with respect to preferred embodiments thereof, it will be understood by those skilled in the art that the foregoing and other changes in forms and details may be made without departing from the spirit and scope of the present invention. It is therefore intended that the present invention not be limited to the exact forms and details described and illustrated, but fall within the scope of the appended claims.

What is claimed is:

1. A polyol-in-oil composition used to transport and stabilize Vitamin C, said composition comprising 0.5 wt % to 5 wt % of at least one emulsifier, 0.1 wt % to 5 wt % of at least one stabilizer, 0.01 wt % to 20 wt % of Vitamin C, 10 wt % to 40 wt % of at least one oil component and 40 wt % to 80 wt % of at least one polyol, each based on the mass of the whole composition, wherein the amount of water in the whole composition is less than 1 wt % and wherein the Vitamin C is dissolved in the polyols.

2. The composition according to claim 1, wherein the proportion of water in the whole composition is less than 0.1 wt %.

3. The composition according to claim 1, wherein the content of Vitamin C in said composition is 5 wt % to 15 wt %.

4. The composition according to claim 1, wherein said at least one emulsifier is selected from the group containing: Cetyl PEGIPPG-10/1 dimethicone or a combination of poly glyceryl alkyl esters.

5. The composition according to claim 4, wherein the combination of poly glyceryl alkyl esters is chosen from the combination of poly glyceryl alkyl esters containing 1 portion by weight of polyglyceryl-4 diisostearate/polyhydroxystearate/sebacate and 0.25 to 4 portion by weight of polyglyceryl-4 stearate, or 1 portion by weight of diisostearoyl polyglyceryl-3 dimer dilinoleate and 0.25 to 4 portion by weight of polyglyceryl-4 stearate.

6. The composition according to claim 1, wherein said at least one stabilizer is chose from nanometer sized or micrometer sized particles of hydrophobic bentonite or hydrophobic silica.

7. The composition according to claim 6, wherein said at least one stabilizer is hydrophobic silica, said hydrophobic silica having a BET surface area from 200 m²/g to 300 m²/g and wherein its methanol wettability is from 50 to 70.

8. The composition according to claim 1, wherein said at least one oil component comprises silicones, fatty hydrocarbons, fatty alcohols and/or fatty esters.

9. The composition according to claim 8, wherein the silicones are chosen from cyclomethicone, dimethicone, and silicone copolymer or mixtures thereof, and the fatty hydrocarbons, fatty alcohols and/or fatty esters are chosen from paraffin oil, polyisobutene, cetyl alcohol, stearyl alcohol, octyldodecanol, cetearyl isononanoate, cetyl ethylhexanoate, cetyl ricinoleate, caprylic/capric triglyceride, decyl oleate, diethylhexyl carbonate, decyl cocoate, isocetyl palmitate, cetearyl ethylhexanoate, isopropyl myristate, myristyl myritate, octyl palmitate, octyl stearate, isopropyl palmitate, triisostearin, steryl heptanoate, 12-15 alkyl benzoate, PPG-15 stearyl ether, PPG-14 butyl ether, PPG-3 myristyl ether, PPG-11 stearyl ether or mixtures thereof.

10. The composition according to claim 1, wherein said at least one polyol is a monohydric, dihydric, and/or trihydric alcohol comprising less than 5 carbons.

11. The composition according to claim 8, wherein the at least one polyol is selected from glycerin, propylene glycol, 1,3-butylene glycol and ethanol.

12. A process of producing the polyol-in-oil composition according to claim 1, which comprises the steps:
a.) mixing all oil components, emulsifiers and stabilizers to obtain a homogeneous phase,
b.) adding Vitamin C to one or more polyols to provide a mixture and heating up the mixture up to a temperature above room temperature until Vitamin C dissolves completely and a homogeneous mixture including a polyols phase is obtained which is then brought down to room temperature, and
c.) adding the polyols phase obtained in step b.) to the oil phase obtained in step a.) and then homogenizing the mixture to obtain the polyol-in-oil emulsion with said content of Vitamin C.

13. The process according to claim 12 comprising the steps of:
a.) mixing all oil components, emulsifiers and stabilizers at 20° C.-30° C. under agitation to obtain a homogeneous phase,
b.) adding Vitamin C into polyols and heating up this mixture up to 90° C. to 100° C. until Vitamin C dissolves and a homogeneous mixture including a polyols phase is obtained which is then brought down to room temperature, and
c.) adding the polyols phase obtained in step b.) slowly into the oil phase obtained in step.a.) under stirring, and then homogenizing the mixture under higher stirring speed obtaining the polyol-in-oil emulsion with a content of said Vitamin C.

14. The process according to claim 12, wherein after the dissolution of the Vitamin C in the polyols phase in step b.) and before step c.) one or more alcohols having a boiling point below the maximum process temperature of step b.) is added to the polyols mixture.

15. A cosmetic product comprising a polyol-in-oil composition according to claim 1.
